(19) United States
(12) Patent Application Publication
(10) Pub. No.: US 2009/0080679 A1
(43) Pub. Date: Mar. 26, 2009

(54) Securing device with light source for a hearing apparatus and corresponding hearing apparatus

(75) Inventor: Uwe Rass, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL INSTRUMENTS PTE. LTD.

(21) Appl. No.: 12/283,406

(22) Filed: Sep. 11, 2008

(30) Foreign Application Priority Data
Sep. 21, 2007 (DE) 10 2007 045 316.9

(51) Int. Cl. H04R 25/00; A61L 2/10

(52) U.S. Cl. 381/322; 422/186.3

(57) ABSTRACT

A securing device for securing a hearing apparatus is provided which has at least one light source, in particular a UV emitter, for emitting light and for irradiating the hearing apparatus that is to be secured. The securing device can be installed in a charger or a storage box, for example, so that the hearing apparatus can be conveniently cleaned there by means of the light. To improve the cleaning effect, the hearing apparatus can be coated at least partially with a photocatalyst.

### CROSS REFERENCE TO RELATED APPLICATIONS

This application claims priority of German application No. 10 2007 045 316.9 DE filed Sep. 21, 2007, which is incorporated by reference herein in its entirety.

### FIELD OF INVENTION

The present invention relates to a securing device for securing a hearing apparatus. In particular the present invention relates to a container or a charger device having a securing device of said kind. In addition the present invention also relates directly to a hearing apparatus. The term “hearing apparatus” is understood in this context to mean in particular a hearing device, but also includes any other sound output device that can be worn on or in the ear, such as, for example, a headset, earphones and the like.

### BACKGROUND OF INVENTION

Hearing devices are portable hearing apparatuses which are used to provide hearing assistance to the hearing-impaired. In order to accommodate the multiplicity of individual requirements, different designs of hearing devices are provided, such as behind-the-ear (BTE) hearing devices, hearing devices with external earpiece (RIC: receiver in the canal), and in-the-ear (ITE) hearing devices, e.g. including full-shell/half-shell (concha) or canal hearing devices (ITE, CIC). The hearing devices cited by way of example are worn on the outer ear or in the auditory canal. In addition to these, however, bone conduction hearing aids as well as implantable or vibrotactile hearing aids are also available on the market. With these, the damaged hearing is stimulated either mechanically or electrically.

Essential components of hearing devices include in principle an input transducer, an amplifier and an output transducer. The input transducer is typically a receiving transducer, e.g. a microphone, and/or an electromagnetic receiver, e.g. an induction coil. The output transducer is mostly realized as an electroacoustic converter, e.g. a miniature loudspeaker, or as an electromechanical converter, e.g. a bone conduction receiver. The amplifier is usually integrated into a signal processing unit. This basic configuration is shown in FIG. 1 by way of the example of a behind-the-ear hearing device. One or more microphones 2 for recording ambient sound are incorporated in a hearing device housing 1 that is designed to be worn behind the ear. A signal processing unit 3, which is likewise integrated into the hearing device housing 1, processes the microphone signals and amplifies them. The output signal of the signal processing unit 3 is transmitted to a loudspeaker or receiver 4, which outputs an acoustic signal. In certain cases the sound is transmitted to the ear drum of the hearing device wearer via a sound tube which is secured in the auditory canal by means of an otoplastic. The hearing device and in particular the signal processing unit 3 are supplied with power by means of a battery 5 which is likewise integrated into the hearing device housing 1.

Hearing devices are exposed to water, perspiration and all kinds of environmental contamination. Cleaning the hearing devices with water or indeed cleaning agents is no straightforward matter on account of the sensitive electronics, the microphones and the receivers. Daily cleaning would, however, be desirable for reasons of hygiene.

In the prior art plastic parts of hearing devices, e.g. earmolds, are usually cleaned at more or less lengthy time intervals in the course of a visit to an acoustician. In addition, cleaning kits are also known which comprise brushes, cloths, etc., and with which the hearing device users can clean their hearing devices themselves.

A method and an apparatus for cleaning hearing aid devices are also known from the publication U.S. Pat. No. 7,182,820 B2. The apparatus comprises a compartment in which the hearing instrument is dried by means of a combination of a heating element and a desiccant.

Also known from the Degussa Science Newsletter “elements” 03/2003, page 8, are titanium oxide-based anti-soiling coatings that are photocatalytically active and hydrophobic. The coating is activated through exposure to UV light and atmospheric humidity, and has a photo-oxidative effect with regard to organic pollutants. As a result, fatty substances that deposit on the respective material are disintegrated to carbon dioxide and water.

The publication WO 2007/066908 A1 discloses a hearing aid with sterilizing charger device. The charger device is equipped with a UV LED or UV lamp in order to sterilize a hearing aid device for public use.

The publication US 2007/0207071 A1 also discloses a hearing device dehumidifier with disinfectant chamber. The sterilization is achieved not by means of UV light, but by means of ozone. This means that internal spaces of the hearing aid can also be sterilized.

### SUMMARY OF INVENTION

An object of the present invention is to facilitate and improve the cleaning of hearing apparatuses for users.

This object is achieved according to the invention by means of a securing device as claimed in an independent claim. A securing device has a retaining element serving to retain the hearing apparatus and made of UV-transparent plastic or glass. As a result the UV light can reach practically all parts of the hearing apparatus. The retaining element constitutes a light conductor which decouples the light in the direction of the hearing apparatus that is to be retained. The retaining element is thus endowed with a dual functionality, i.e. serving to guide the light in addition to the customary retaining function.

By cleaning with light, in particular UV light, the electronics, microphones and receivers of hearing devices in particular can advantageously be prevented from being damaged during cleaning. At the same time a very high cleaning effect, in particular with regard to bacteria, is achieved by means of exposure to (UV) radiation.

The at least one light source preferably consists of a UV LED. LEDs of this kind have a very high level of efficiency and at the same time possess the small dimensions that are necessary particularly in the hearing device field.

It is particularly advantageous if a container for storing the hearing apparatus, e.g. a storage box for hearing devices, is equipped with a securing device of said kind. This allows the hearing apparatus to be conveniently cleaned when not in use, without the need for an additional device.

It can also be favorable if a charger for charging the hearing apparatus has the aforesaid securing device with light source. In this case the energy supply for charging the hearing apparatus can also be used simultaneously for the light source.

Furthermore the container or, as the case may be, the charger can have a lid into which a plurality of UV LEDs are integrated. This will enable exposure to UV radiation to be realized also from the top of the device.

In addition it can be advantageous if the inner surface of the container for storing the hearing apparatus is at least partially mirrored. In this way exposure to (UV) radiation can be realized on all sides.

The aforesaid object of enabling a hearing apparatus to be cleaned more easily and efficiently is also achieved by means of a hearing apparatus which is designed to be worn in or on the ear and which is coated on at least a part of its outer surface with a photocatalyst. More particularly, the photocatalyst can contain titanium dioxide. During (UV) irradiation the photo-oxidative effect with regard to organic substances is then used for cleaning.

### BRIEF DESCRIPTION OF THE DRAWINGS

The present invention is explained in more detail with reference to the attached drawings, in which:

FIG. 1 shows the basic configuration of a hearing device according to the prior art;

FIG. 2 shows a cross-section through a storage box according to the present invention, and

FIG. 3 shows a cross-section through a light conductor retaining element.

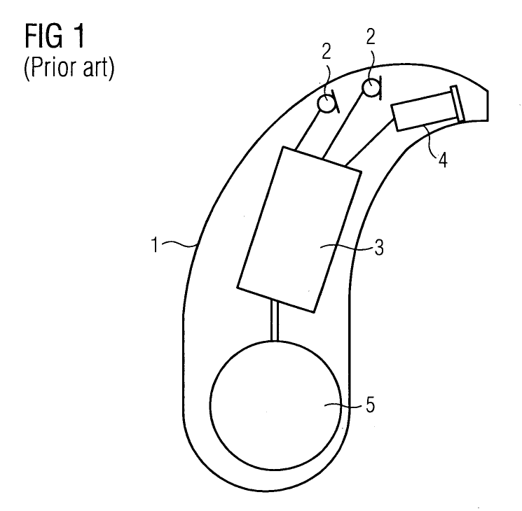

### DETAILED DESCRIPTION OF INVENTION

The exemplary embodiments described in more detail below represent preferred embodiment variants of the present invention.

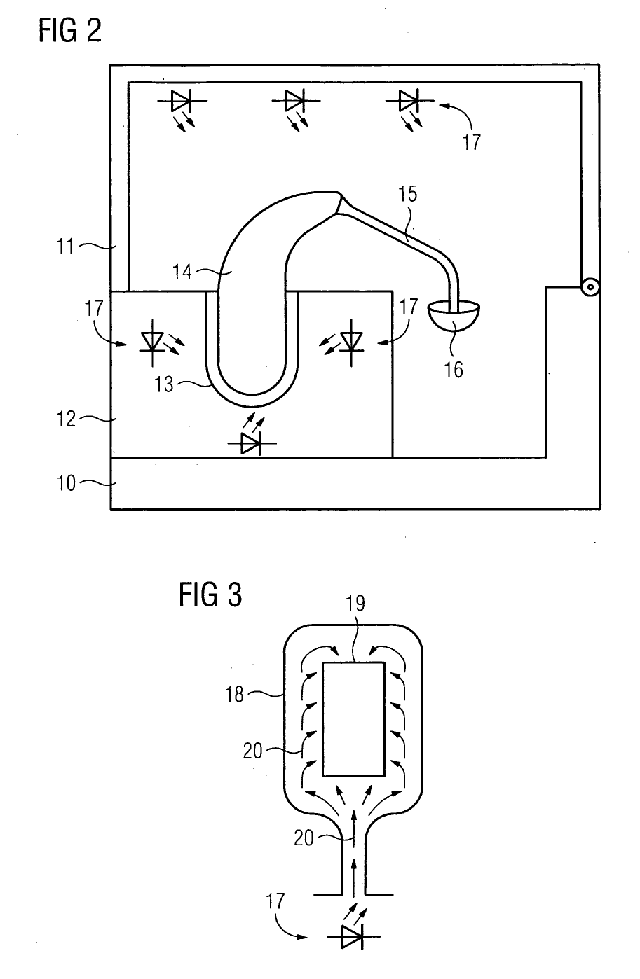

For cleaning purposes, a hearing apparatus, in this case a hearing device, is irradiated with visible light or UV light in a securing device. In the example of FIG. 2 the securing device is integrated into a storage box which has a tray 10 and a lid 11 pivotably attached thereto. In this case the securing device is symbolized by a cuboid 12 having a recess 13. The hearing device 14 that is to be stored and cleaned is inserted into the recess 13. It projects from the recess 13 with a part of the hearing device housing as well as the receiver tube 15 attached thereto and the earmold 16. It can therefore be easily inserted into the recess 13 and removed again.

If the storage box is simultaneously used as a charger, the cuboid 12 contains the corresponding charging device and contacts are provided in the recess 13 which, with the hearing devices in the inserted state, are connected to corresponding contacts of the hearing device (not shown in FIG. 2). In the case of a pure charger, for example, only the cuboid 12 with the recess 13 may be provided as a securing device with integrated charging device but without the tray 10 and the lid 11.

According to the invention a plurality of UV emitters, possibly also just one, are then arranged in, on or close to the securing device. In the example shown in FIG. 2 the UV emitters are UV LEDs 17. In the arrangement shown they are disposed both in the cuboid 12 and on the inside of the lid 11. This means that the hearing device that is to be stored or, as the case may be, charged can be irradiated with UV light from all sides. The cuboid 12, i.e. the retainer for the hearing device 14, is in this case manufactured from UV-transparent plastic or glass. This means that when emitted from the UV LEDs 17 integrated into the retainer, but also from the UV LEDs 17 mounted on/in the lid 11, the UV light can also easily reach those points of the hearing device 14 or hearing device housing which are physically in contact with the retainer in the recess 13.

Commercially available UV LEDs can be used as UV emitters. Where appropriate, their UV ranges are chosen with regard to optimal bactericide or oxidative effect.

The effect of the UV light can be reinforced by means of special catalysts. Thus, it is favorable to coat the housing of the hearing device 14, the sound tube 15 and/or the earmold 16 with a catalyst of said kind. An example of a suitable photocatalyst of said kind is titanium dioxide (TiO₂), which is used as white pigments in paints. In practice, therefore, a TiO₂-containing coating is applied to the hearing device and/or its components.

A further embodiment variant of an inventive securing device for hearing devices is shown schematically in FIG. 3. In this case the device is a light conductor frame 18 which has in its center a cutout 19 for introducing or retaining a hearing device. The cuboid 12 from FIG. 2 can also be implemented as a light conductor, such that the schematic shown in FIG. 3 would represent a view from above onto the cuboid 12, with the cutout 19 corresponding to the recess 13.

The light of a UV LED 17 is then guided in the direction of the cutout 19 in accordance with the arrows 20 shown in FIG. 3. The light is uniformly distributed around the cutout 19 and is therefore decoupled in it in the direction of the inserted hearing device (not shown).

Mirroring the surfaces in the interior of the charger or, as the case may be, storage box provides a further possibility of distributing the (UV) light. As a result of the mirroring it is possible to irradiate the entire surface or at least a large part thereof with (UV) light using one UV emitter or a small number of UV emitters without a substantial portion of said light being absorbed at the surface of the storage box.

1.-8. (canceled)

9. A securing device for securing a hearing apparatus, comprising:
a UV light source for irradiating the hearing apparatus to be secured; and
a retaining element for retaining the hearing apparatus made of UV-transparent plastic or glass, wherein the retaining element has a light conductor which decouples the UV light in the direction of the hearing apparatus that is to be retained.

10. The securing device as claimed in claim 9, wherein the light conductor is the retaining element.

11. The securing device as claimed in claim 9, wherein the light source is a UV LED.

12. The securing device as claimed in claim 9, wherein the securing device has a plurality of light sources.

13. A device, comprising:
a securing device for securing a hearing apparatus, having:
a UV light source for irradiating the hearing apparatus to be secured, and
a retaining element for retaining the hearing apparatus made of UV-transparent plastic or glass, wherein the retaining element has a light conductor which decouples the UV light in the direction of the hearing apparatus that is to be retained.

14. The device as claimed in claim 13, wherein the device is a container for storing the hearing apparatus, into which the securing device is integrated.

15. The device as claimed in claim 13, wherein the device is a charger for charging the hearing apparatus, wherein the securing device is a part of the charger.

16. The device as claimed in claim 14, further comprising a lid into which a plurality of UV LEDs are integrated.

17. The device as claimed in claim 15, further comprising a lid into which a plurality of UV LEDs are integrated.

18. The device as claimed in claim 14, wherein the inner surface of the container is at least partially mirrored.

19. The device as claimed in claim 16, wherein the inner surface of the container is at least partially mirrored.

20. The device as claimed in claim 14, wherein a hearing apparatus is placed in the container.

21. The device as claimed in claim 20, wherein the hearing apparatus is coated on at least a part of its outer surface with a photocatalyst.

22. The device as claimed in claim 21, wherein the photocatalyst contains titanium dioxide.

23. The device as claimed in claim 15, wherein a hearing apparatus is charged via the charger, wherein the hearing apparatus is coated on at least a part of its outer surface with a photocatalyst.

24. The device as claimed in claim 23, wherein the photocatalyst contains titanium dioxide.

25. A hearing apparatus designed to be worn in or on an, wherein the hearing apparatus is coated on at least a part of its outer surface with a photocatalyst.

26. The hearing apparatus as claimed in claim 22, wherein the photocatalyst contains titanium dioxide.
